(19) United States
(12) Patent Application Publication
(10) Pub. No.: US 2020/0275618 A1
(43) Pub. Date: Sep. 3, 2020

(54) PLANT-SYSTEM WITH INTERFACE TO MYCORRHIZAL FUNGAL COMMUNITY

(71) Applicant: GAMAYA SA, Morges (CH)

(72) Inventors: Yosef AKHTMAN, St-Prex (CH); Vaia SARLIKIOTI, Epalinges (CH)

(21) Appl. No.: 16/649,844

(22) PCT Filed: Sep. 21, 2018
(86) PCT No.: PCT/IB2018/057308

§ 371 Date: Mar. 23, 2020

(30) Foreign Application Priority Data
Sep. 21, 2017 (IB) PCT/IB2017/055725

(51) Int. Cl. A01G 24/20; A01G 18/10; A01G 7/06; A01G 27/00; A01M 21/04; G01N 33/00

(52) U.S. Cl. CPC A01G 24/20; A01G 18/10; G01N 33/0098; A01G 27/003; A01M 21/043; A01G 7/06

(57) ABSTRACT

A plant system (100), comprising a plurality of plants (102) in a substrate (101); a mycorrhizal fungal community (104) in the substrate arranged to form a biological interface with roots of the plants enabling an exchange of chemical substances between the fungus and the plurality of plants; and at least a sensor (207), which interfaces with said mycorrhizal fungal community and is configured to collect sensory information about a physiological condition and phenotypic state of said plurality of plants.

### FIELD OF INVENTION

The presently disclosed embodiments are related, in general, to a method and apparatus of sensory interface between a computing system and a community of living plants. More specifically a system is described which enables controlled exchange of electrical signals, chemical substances, and sensory information between an artificial computing device and a plurality of plants, as well as the generation of electrical energy by means of bioelectrochemical reaction involving a mycorrhizal network.

### BACKGROUND OF INVENTION

Agriculture and food production are a vital part of the global economy. The world population is expected to grow from 7.2 billion today to 10 billion by 2050. This presents a widely recognised need for farmers to increase food production by enhancing efficiency, and protect farmland and the environment. The mission of technological innovation in agriculture is to increase the efficiency and sustainability of large industrial farming and the productivity and scalability of small-holder farming. Large industrial farms have tremendous throughput which they currently achieve through extensive use of chemical agents including fertilisers, pesticides and herbicides. At such large quantities, these chemicals degrade the land, damage the environment, and endanger the farmers' health, thereby presenting a very serious challenge to responsible land use. Small-holder farmers often use better land management practices and lower amounts of chemicals but do not produce enough yield.

To address their respective challenges, farmers require reliable and precise tools to monitor, diagnose and consequently optimize their use of agricultural land, applying customized amounts of inputs to small sub-field areas. This concept is known as precision agriculture.

Existing innovation in agriculture relies on advanced agricultural machinery and automation. This is evidenced by self-driving tractors with integrated on-board computers, GPS and sensors. Machinery with variable rate dosing of fertilizers and chemicals, yield mapping and automatic and precise seeding is a standard among advanced agricultural producers. Providing these automated machines with precise prescriptions of what to apply, at which amounts, in which subsections of the field requires labor-intensive human input. Agronomists perform manual inspections of the farmland and collect soil and plant samples by hand for analysis in the laboratory. This hands-on approach allows for in-depth knowledge of the fields but is too labor-intensive to scale past several hectares without large teams of agronomists. Lab analysis is also too slow for the rapid responses needed to effectively address plant stress, where responding quickly is paramount. Without quick, accurate, and precise information about the health of sub-field regions, farmers apply the same amount of inputs to the entire field.

Currently, both data collection but also treatment of the plants is highly resource, energy and time consuming. Electric sensors, cables or wireless data transmission devices, and computers are necessary to collect the data. These systems are costly but also susceptible to interruption, often through agricultural practices. This is likewise for treatment of plants if relying on either treatment of the seed or plants by mechanical applications. So far the performance and physiological stage of a plurality of plants in larger field has to be monitored by positioning multiple sensors and connecting them by wire or wireless systems to analytical systems. This is highly cost and labor intensive. There is an unmet need to reduce the costs and hardware-decency of these conventional systems.

Plants establish symbiotic relationships with other organisms to increase their competitiveness for environmental resources. Most of these relationships are developed between the plant root system and soil organisms and result in the increase of the water and nutrient uptake efficiency. The symbiotic organisms to the plant root belong to two main groups: 1. Bacteria and 2. Fungi with the former group being the most well studied.

Symbiotic bacteria known as rhizobia are species specific and are commonly found in the leguminous family. The bacteria create pods on the inside of the roots that lead to N2-fixation providing the plant the necessary N for its development (Pawloski, 2009). It has been proven that rhizobia play a significant role in improving the soil fertility in poor soils as well as under salt, heat and acid stress as well as under the effect of heavy metals (Zahran, 1999). Rhizobia and roots are highly host specific as it has been found that they exchange chemical signals between partners.

Fungi symbiotic relationship on the other hand are not species specific. Arbuscular mycorrhizas, as the fungi that penetrate the cortical cells of the roots are called, represent 10% of the soil biomass, playing a significant role in the nutrient exchange capacity between the root and the soil. In particular, they offer the plant a significantly extended root system to transfer nitrogen, phosphorus and water from distant locations in exchange for sugars produced by the plant through photosynthesis. Chen et al., (2016) showed that the symbiotic relationship between arbuscular fungi and 6 tree species can increase the foraging accuracy up to 201% and foraging precision up to 130%. This increase in foraging accuracy and precision is especially important on soils with an uneven nutrient distribution.

In addition to the increased nutrient uptake efficiency, the Arbuscular Mycorrhizal Fungi have been shown to bear significant and diverse positive effects on the development of plants including the improved resilience to abiotic stresses, such as draught and salinity, as well as primed immunity to biotic stresses, including disease, pests, and microbial pathogens (Aroca 2013).

An effort to harvest the positive impact of the above described symbiotic relationships is reflected in fertilization focused patent publications. Different combinations of fungi and bacteria are created together aiming to increase the agricultural productivity with their application before or during the cultivation season. These products target a variety of plant species grown both on field and under greenhouse conditions (AU2017201009, US20170107160, US20170020138, PCT/US2016/043933).

### SUMMARY OF INVENTION

The present invention solves the above described problem, by utilizing the natural network provided by mycorrhizal fungi to communicate plant signals to a computer system. In a preferred embodiment these plant signals are used to directly trigger a treatment of the plants in response to the signal.

In a first aspect, the invention provides a plant system, comprising a plurality of plants in a substrate; a mycorrhizal fungal community in the substrate arranged to form a biological interface with roots of the plants enabling an exchange of chemical substances between the fungus and the plurality of plants; and at least a sensor, which interfaces with said mycorrhizal fungal community and is configured to collect sensory information about a physiological condition and phenotypic state of said plurality of plants.

In a preferred embodiment, the sensor is continuously interfacing with the mycorrhizal fungal community and able to detect chemical or biochemical substances without disrupting said community.

In a further preferred embodiment, the sensor is based on a method selected from a list consisting of mass spectroscopy, Raman spectroscopy, SL biosensor, fluorometer, MEMS sensor, pH sensor, amperemeter.

In a further preferred embodiment, the sensor is linked to a computer converting the sensory information into a recommendation for action or action.

In a further preferred embodiment, the action is selected from the group consisting of treating the plants with chemical or biochemical substances, irrigation, harvesting, release of biocontrol agents such as any one from the list comprising herbicides, pesticides, nematicides, fungicides, application of strigolactones and Myc factors, targeted distribution of macro nutrient, targeted distribution of micro nutrient such as any one from the list comprising phosphorus (P), nitrogen (N), potassium (K), calcium (ca), magnesium (Mg), iron (Fe), manganese (Mn), zinc (Zn), copper (Cu), topping, application of growth regulators.

In a further preferred embodiment, the system further comprises a reactor configured to enable a release of chemical or biochemical substances into the mycorrhizal fungal community to the plurality of plants rooted in a substrate.

In a further preferred embodiment, the system further comprises at least a bioelectrochemical module configured to generate electrical current for powering a bioelectrochemical plant-computer interface system.

In a further preferred embodiment, the substrate is enabled to support growth and development of at least one of the plurality of plants. The substrate is one of the list comprising at least a substrate enclosed in an artificial container; a substrate that forms a part of an agricultural field; and a natural substrate that is part of a natural vegetative habitat.

In a further preferred embodiment, the substrate is inoculated with mycorrhizal fungal community configured to permeate parts, or the entirety of a system's usable volume.

In a further preferred embodiment, the reactor is a bioelectrochemical reactor comprising at least one semi-isolated internal container that contains at least a chemical substance that is of physiological or nutritional significance to the plurality of plants.

In a further preferred embodiment, the bioelectrochemical reactor comprises at least one semi-isolated internal container that contains at least a chemical substance that is of physiological or nutritional significance to the plurality of plants; and the mycorrhizal fungal community forms a mycelium fiber network that permeates the container and carries the at least one chemical substance to the roots of a plurality of plants.

In a further preferred embodiment, the system further comprises a computational and communication device configured to record, process and communicate a plurality of analog and digital electrical signals. The computational and communication device comprises a radio interface enabled to transmit digital information to intended other computational devices, or an intended centralized base-station.

In a further preferred embodiment, at least one sensor is connected to the computation and communication device by means of conductive wires; and the results of collected sensory information being transmitted to the computational and communication device in the form of analog or digital electric signals.

In a further preferred embodiment, a plurality of the at least one internal containers form a biochemical power cell that generates electric current that is utilized to power computational and communication devices and at least one sensor. A first of the plurality of internal containers is configured to generate an excess of cations and free electrons through biochemical reaction involving hyphae of the mycorrhizal network. A second of the plurality of internal containers is configured to absorb cations and free electrons through biochemical reaction involving hyphae of the mycorrhizal network. The system further comprises a cation transport membrane that separates the first internal container and the second internal container and is enabled to allow for the transport of ions between the two respective first and second internal containers; and a pair of electrodes connected by a conductive wire and a load that allows for a transport of electrons between the first and second internal containers.

In a further preferred embodiment, the plurality of plants is comprising plants selected from the group consisting of annual, and perennial plants.

In a further preferred embodiment, the plant is am annual field crops selected from the group consisting of maize, soy, wheat, rice, barley, rapeseed, canola, tobacco and sunflower.

In a further preferred embodiment, the plant is a vegetable selected from the group of tomato, pepper, melon, squash, cucumber, pumpkin, peas, beans, broccoli, cauliflower, cabbage, Brussel sprouts.

In a second aspect, the invention provides a method for improving the physiological state of a plurality of plants. The method comprises the steps of
(i) planting a plurality of plants in a substrate which comprises a mycorrhizal fungal community arranged to form a biological interface with roots of the plants enabling an exchange of chemical substances between the fungus and the plurality of plants;
(ii) collecting sensory information about a physiological condition and phenotypic state of said plurality of plants using at least one sensor, which interfaces with said mycorrhizal fungal community; and
(iii) analyzing said collected sensory information to trigger or suggest an action to improve the physiological state of said plurality of plants.

In a further preferred embodiment, the sensory information are analyzed to detect biotic stress, abiotic stress, nutrient deficiency or plant phenology.

In a further preferred embodiment,

(i) the biotic stress is selected from the group comprising pressure by competing, parasitic or harmful plants, pests, fungi or microorganisms;
(ii) the abiotic stress is selected from the group comprising heat, cold, drought, flooding, salinity, acidification, soil compaction and physical damage;
(iii) the nutrient deficiency is selected from the group comprising phosphorus (P), nitrogen (N), potassium (K), calcium (Ca), magnesium (Mg), iron (Fe), manganese (Mn), zinc (Zn), and copper (Cu) deficiency; and/or
(iv) the plant phenology is selected from the group comprising maturity, leaf senescence, flowering, seed protein content, and seed water content.

In a further preferred embodiment, the sensory information is established by measuring chemical, biochemical compounds, or other signal transported or conveyed by the mycorrhizal fungal community.

In a further preferred embodiment, the biochemical, chemical compound, or other signal is selected from the group consisting of strigolactones, plant hormones, nitrate reductase, glutamate synthetase, glutamate synthase, ethylene, jasmonic acid, salicylic acid, Myc factors, genistein, lipochitooligosaccharides, phosphatases, pH gradient, or electrical current.

In a further preferred embodiment, the sensor is selected from the group consisting of SL biosensors, fluorometers, MEMS sensors, pH sensor; amperemeter.

In a further preferred embodiment, the action is triggered automatically by a computer system.

In a further preferred embodiment, the triggered or recommended action is selected from the group consisting of

(i) release of biocontrol agents selected from the group consisting of herbicides, pesticides, nematicides, fungicides, or other bioactive compounds;

### (ii) Irrigation;

(iii) Application of strigolactones AM symbiosys;
(iv) Targeted distribution of macro and micro nutrients selected from the group consisting of phosphorus (P), nitrogen (N), potassium (K), calcium (Ca), magnesium (Mg), iron (Fe), manganese (Mn), zinc (Zn), and copper (Cu);
(v) Agronomic operations selected from the group consisting of topping, application of growth regulators, harvesting.

In a third aspect, the invention provides a software product which when loaded and executed by a computer, enables the computer to implement the method steps of any one of the methods and preferred embodiment thereof as described herein above.

### BRIEF DESCRIPTION OF DRAWINGS

Further novel properties and advantages of the proposed invention will become apparent to one of skill in the art, through the detailed description of example preferred embodiments as set forth in the remainder of the present application and with reference to the drawings.

The accompanying drawings illustrate the example preferred embodiments, and other aspects of the invention. Any person with ordinary skills in the art will appreciate that the illustrated element boundaries (e.g., boxes, groups of boxes, or other shapes) in the figures represent one example of the boundaries. In some examples, one element may be designed as multiple elements, or multiple elements may be designed as one element. In some examples, an element shown as an internal component of one element may be implemented as an external component in another, and vice versa. Further, the elements may not be drawn to scale.

Various embodiments will hereinafter be described in accordance with the appended drawings, which are provided to illustrate and not to limit the scope in any manner, wherein similar designations denote similar elements, and in which:

FIG. 1 illustrates a plant system n accordance with an example embodiment, and its core components, including an enclosure; a substrate; a group of plants; a mycelium network;

FIG. 2 provides further details on the design of the bioelectrochemical reactor and its main components, particularly biochemical reaction chambers and a plurality of embedded sensors;

FIG. 3 exemplifies three of possible reactions that can take place in the bioelectrochemical reactor, namely an uptake of potassium, ammonium, phosphorus and nitrogen by an hyphae of the mycelium network;

FIG. 4 details a generation of electric current by a bioelectrochemical reaction occurring in the bioelectrochemical reactor;

FIG. 5 illustrates a network of a plurality of the bioelectrochemical systems interconnected by means of ad-hoc, or cellular wireless communications network with the aim of process monitoring, as well as aggregation and processing of the acquired information;

FIG. 6 illustrates an agricultural input delivery system that performs targeted delivery of fertilizers and crop protection chemicals to a field of agricultural crops utilizing a network of bioelectrochemical reactors organized into an interconnected network;

FIG. 7 contains a flowchart illustrating a method for improving the physiological state of a plurality of plants according to an example embodiment of the invention; and

FIG. 8 illustrates a plant system and a bioelectrochemical module in accordance with an example embodiment, and its core components, including an enclosure; a substrate; a group of plants; a mycelium network; a bioelectrochemical reactor; a power module; a computing device; and a data communications module.

### DETAILED DESCRIPTION OF THE PREFERRED EMBODIMENTS

The present invention will be better understood with reference to the detailed description of preferred embodiments and figures set forth herein. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example”, “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

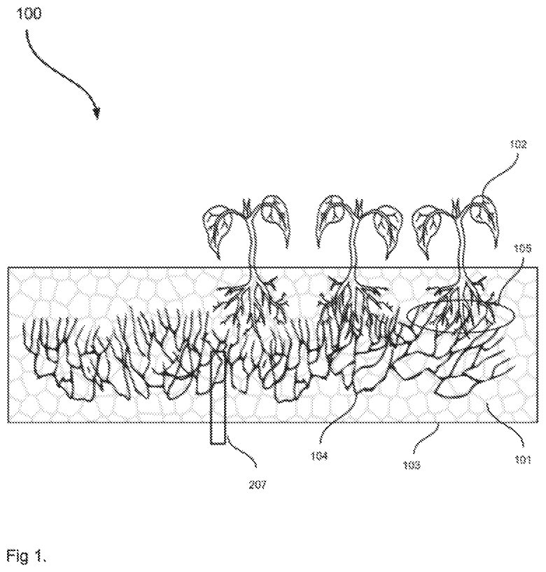

According to embodiments illustrated herein, a plant system 100 depicted in FIG. 1 is disclosed. The plant system 100 comprises a substrate 101 that is able to support the growth and development of one or multiple plants 102. In one embodiment, an example of the substrate 101 is a natural fertile soil. The substrate 101 is either enclosed in an artificial container 103, or is open and constitutes a section of an agricultural field, or a natural vegetative ecosystem (not illustrated in FIG. 1).

The substrate 101 is inoculated with mycorrhizal fungus community 104 that permeates part or the entirety of its volume. In particular, a mycelium network 104 produced by the mycorrhizal fungus community creates a biological interface 105 with roots of the plants 102 enabling an exchange of chemical substances between the mycorrhizal fungus community 104 and the plant 102.

Suitable fungus strains, method of cultivating these strains in vitro and incubation substrate are—for example—described in US 2005/0132431, hereby incorporated by reference.

The substrate can be soil (for example if the method or system of the invention is applied in an open field) or other substrates likes rockwool (if the system is applied in a protected environment like glasshouses).

The mycorrhizal fungus community can be established by inoculation of the substrate. However, it is also possible to utilize a naturally occurring mycorrhizal fungus community.

Suitable mycorrhizal fungus are for example described in WO 2012/038740 A1. The species of mycorrhizal fungus suitable for use in the invention is any species, preferably those which are—in addition to conveying sensory information—capable of augmenting levels of available nutrients in the soil with further organic and inorganic nutrients that are assimilable by a crop plant. Suitable species of mycorrhizal fungus for use in the present invention include those that are capable of colonizing a host plant's roots, either intracellularly as in arbuscular mycorrhizal fungi (AMF), or extracellularly as in ectomycorrhizal (EcM) or ericoid mycorrhizal (EM) fungi.

AMF are mycorrhizae whose hyphae enter into the plant cells, producing structures that are either balloon-like (vesicles) or dichotomously-branching invaginations (arbuscules). The structure of the arbuscules greatly increases the contact surface area between the hypha and the cell cytoplasm to facilitate the transfer of nutrients between them. Examples of genera of AMF of use in the invention include Glomus, Gigaspora, Acaulospora and Sclerocystis. Glomus species are particularly useful.

Suitable species include Glomus fasciculatum, G. intraradices, G. claroideum; G. intra, G. clarum, G. brasilianum, G. deserticola, G. monosporus, G. mosseae, G. tortuosum, G. sinuosum, Gigaspora margarita, Gigaspora gigantean and Acaulospora longular.

Ectomycorrhizae (EcM) are typically formed between the roots of around 10% of plant families, mostly woody plants including the birch, dipterocarp, eucalyptus, oak, pine, and rose families, and fungi belonging to the genera Basidiomycota, Ascomycota, and Zygomycota. Ectomycorrhizae consist of a hyphal sheath or mantle covering the root tip and a hartig net of hyphae surrounding the plant cells within the root cortex. Outside the root, the fungal mycelium forms an extensive network within the soil and leaf litter. Nutrients move between different plants through the fungal network. Genera of EcM of use in the invention include Suillus, Boletus, Lactarius, Laccaria, Pisolithus and Rhizopogon. Examples of species of EcM genera for use in the invention include Pisolithus tictorus, Laccaria laccata, L. bicolor, Rhizopogon villosuli, R. rubescens, R. fulvigleba, R. luteolus, and R. amylopogon.

Ericoid mycorrhizas (EM) are the third of the three more ecologically important types of mycorrhizal fungus that have a simple intraradical (i.e. it grows in cells) phase, consisting of dense coils of hyphae in the outermost layer of root cells. There is no periradical phase and the extraradical phase consists of sparse hyphae that do not extend far into the surrounding soil. Ericoid mycorrhizae are known to have saprotrophic capabilities and these are thought to enable plants to receive nutrients from not-yet-decomposed materials via the decomposing actions of their ericoid partners. A suitable genus of EM of use in the present invention is Pezizella.

Another illustrative embodiment of the invention comprises a mycorrhizal fungi component which is marketed under the trademark MycoApply® by Mycorrhizal Applications Inc., 810 NW E St., Grants Pass, Oreg. 97526.

In addition to Glomus intraradices, Glomus etunicatum, Glomus aggregatum, and Glomus mossae, other Glomus species may be used to make mycorrhizal fungi component of the invention, including the following: G. albidum, G. caledonium, C. claroideum, G. clarum, G. clavispora, G. constrictum, G. coronatum, G. deserticola, G. diaphanum, G. eburneum, G. fragilistratum, G. gerosporum, G. globiferum, G. hadleyi, G. hyalinum, G. insculptum, G. lamellosum, G. luteum, G. macrocarpum, G. manihot, G. microaggregatum, G. mirificum, G. monosporum, G. pustulatum, G. sinuosum, G. spurucum, G. tortuosum, G. verruculosum, G. versiforme, and G. viscosum (available from INVAM-West Virginia University). The following endomycorrhizal species may also be used to make the mycorrhizal fungi component of the invention: Ambisporaceae spp.; Archaeosporaceae spp. [Ar. leptoticha, Ar. gerdemannii, and A. trappei (available from INVAM-West Virginia University)] Geosiphonaceae spp., Acaulosporaceae spp. [A. colossica, A. delicatta, A. denticulate, A. foveata, A. koskei, A. lacunosa, A. laevis, A. longula, A. mellea, A. morrowiae, A. rehmii, A. scrobiculata, A. spinosa, and A. tuberculata (available from INVAM-West Virginia University)]; Enterophosphoraceae spp. (E. colombiana, E. contigua, E. infrequens, E. kentinesis), Dicersisporaeceae spp, Gigasporaceae spp. [including Gi. albida, Gi. decipiens, Gi. gigantea, Gi. margarita, and Gi. rosea) (available from INVAM, West Virginia University)]; Paraglomus spp. (P. brasilianum and P. occultum (available from INVAM-West Virginia University)]; and Scutellospora spp (S. calospora, S. cerradensis, S. coralloidea, S. dipurpurascens, S. erythropa, S. fulgida, S. gregaria, S. heterogama, S. pellucida, S. persica, S. reticulate, S. rubra, S. scutata, and S. verruscosa (available from INVAM-West Virginia University)]. Arbuscular mycorrhizal fungi are naturally occurring soil fungi, and new strains and species may be discovered in the future and used to make the invention.

The plant system 100 further comprises at least a sensor 207, which interfaces with the mycorrhizal fungal community 104 and is configured to collect sensory information about a physiological condition and phenotypic state of the plants 102.

In a preferred embodiment, the sensor 207 is continuously interfacing with the mycorrhizal fungal community 104 and able to detect chemical or biochemical substances without disrupting the community 104.

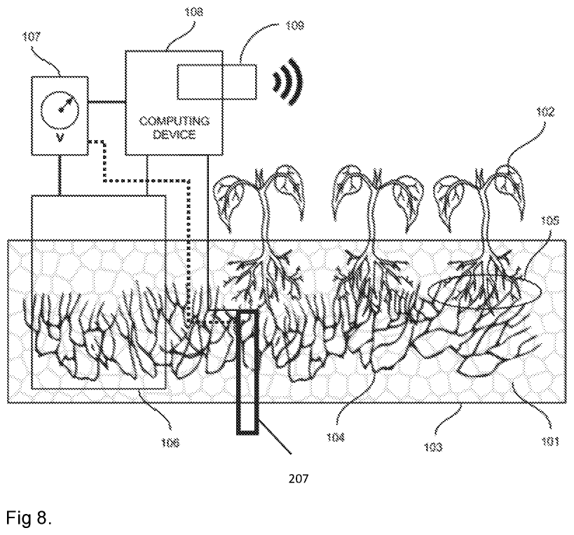

FIG. 8 illustrates a particular example, where the plant system 100 is combined with a number of additional features, The additional features comprise a bioelectrochemical reactor 106 that enables the controlled exchange of chemical substances with an hyphae of the mycelium network 104.

In a further preferred embodiment, the plant system 100 further comprises a computing device 108 that is capable to record, process and communicate a plurality of analog and digital electrical signals transmitted by the bioelectrochemical reactor 106. In particular the sensor 207 is electrically connected to the computing device. Also in particular, the computational device 108 comprises a radio interface 109 that is able to transmit digital information to other computational devices, or a centralized base-station (both not illustrated in FIG. 1).

In a further preferred embodiment, the plant system 100 further comprises a power module 107 that may be configured to collect and store electrical energy generated by the bioelectrochemical reactor 106. Subsequently, the power module 107 may be configured to supply electrical energy to empower the operation of the computing device 108. Optionally the sensor 207 may also be connected to the power module 107, as indicated by the dashed line in FIG. 8.

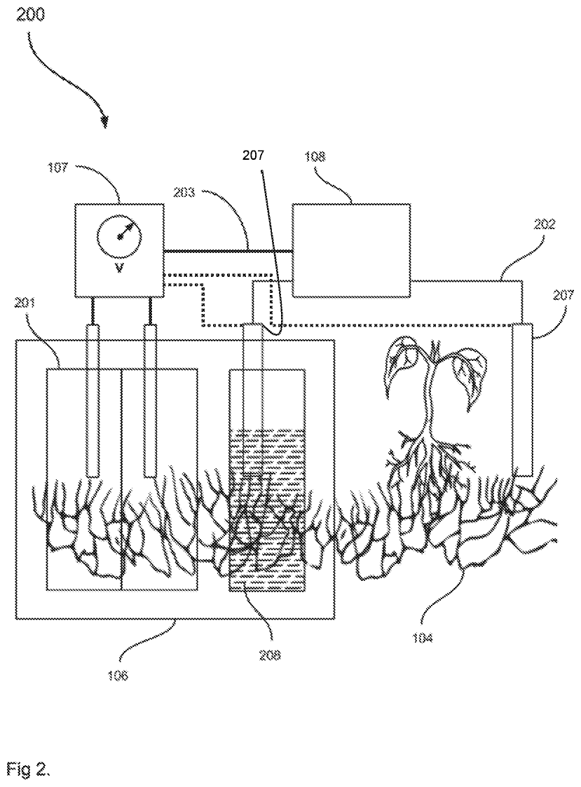

An example embodiment of the bioelectrochemical reactor 106 is further detailed in FIG. 2 and comprises a plurality of semi-isolated containers 201. Each container 201 comprises a substrate (not illustrated in FIG. 2) that is suitable to support the development of the mycelium network—also known as fungal tissue 104. In one embodiment a carbon fabric, or felt is utilized as conductive substrate in containers 201. Additionally, each container 201 carries a particular chemical substance 208 that is of physiological or nutritional significance to the plants 102. Two or more of containers 201 may be further grouped together to produce bioelectrochemical modules that may act as biochemical fuel cells and generate electrical current. The mycelium fiber network 104 permeates the substrate inside the containers 201 and is enabled to carry the corresponding chemical substances to the roots of the plants 102.

The containers 201 further include a plurality of the sensors 207 that may perform a plurality of physical, chemical and biological measurements on the content of the individual containers 201 yielding information concerning the physiological condition and phenotypic state of plants 102. Examples of such measurement include pH levels, moisture content and salinity. Furthermore, the measurements may involve gas and chemical sensors for the detection of a plurality of specific chemical substances, including for example strigolactones and various phytohormones. The signals produced by the sensors 207 may be transmitted to the computing device 108 that is capable of receiving, processing and relaying of these signals to a plurality of the other computing devices, or a centralized server (not illustrated in FIG. 2). Optionally the sensors 207 may also be connected to the power module 107, as indicated by the dashed line in FIG. 2.

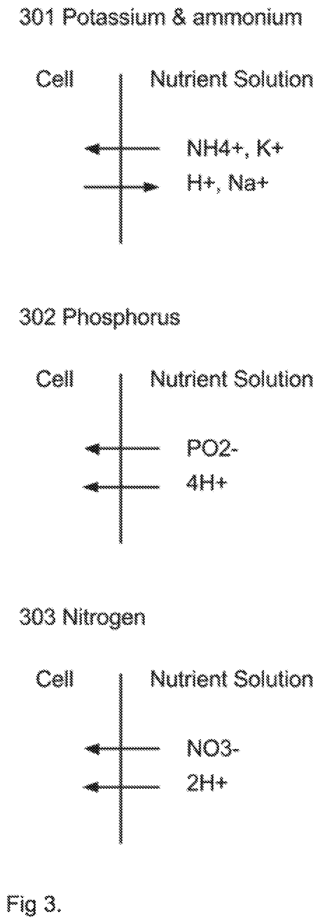

A number of examples of the biochemical reactions that may occur in the plurality of separate containers 201 of the bioelectrochemical reactor 106 are described in FIG. 3. In particular, reaction 301 involves the uptake of positive ions of potassium and ammonium in exchange for the positive ions of hydrogen and natrium. Reaction 302 involves the uptake of negative ions of phosphorus oxide accompanied by the uptake of positive ions of hydrogen. Likewise, reaction 303 involves the uptake of negative ions of nitrogen oxide accompanied by the uptake of positive ions of hydrogen.

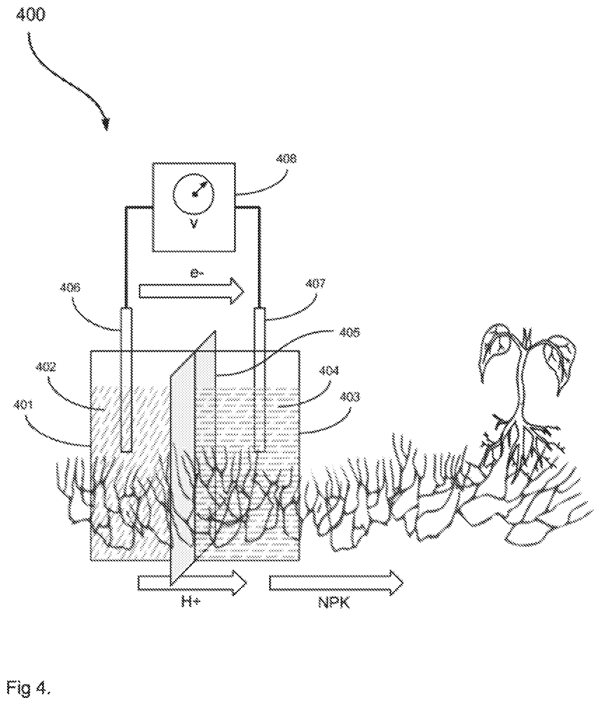

An example embodiment of two containers forming a biochemical fuel cell is further depicted in FIG. 4. Specifically, container 401 accommodates a nutrient solution 402, that produces positively charged hydrogen ions upon uptake by the fungus hyphae resulting in excess of hydrogen cations and free electrons in the container. Container 403 accommodates a nutrient solution 404, that requires a supply of hydrogen cations and free electrons as part of the uptake of the solution molecules by the fungus hyphae. Subsequently, a proton exchange membrane 405 facilitates the transport of hydrogen cations from container 401 into container 403. In one embodiment a carbon fabric, or felt is utilized as conductive substrate in containers 401 and 403.

Furthermore, the excess free electrons generated by the biochemical reaction in container 401 are collected by an anode 406 and are transported to container 403 where they are released by a cathode 407. The resultant electron flux between containers 401 and 403 and through a load 408 produces the desired electrical current.

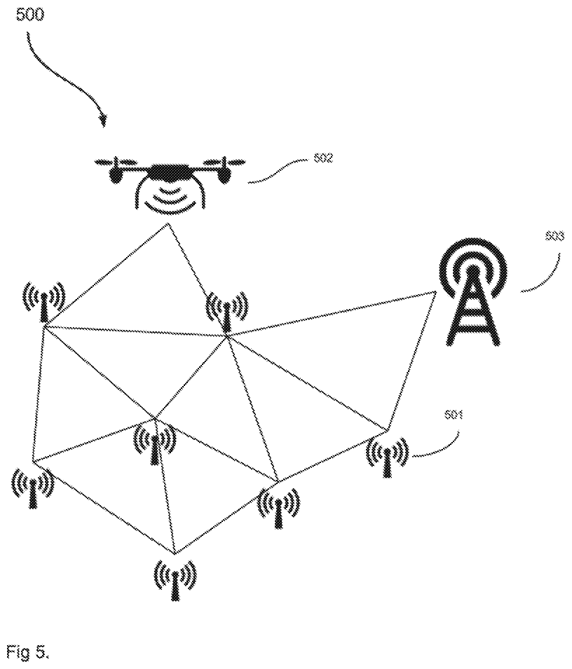

A plurality of plant systems 100 of FIG. 1 may be deployed throughout agricultural farmland or a natural vegetative ecosystem to form a sensor network 500 as shown in FIG. 5. Specifically, the communication units 109 accommodated by the computational devices 108 of each plant system 100 (not shown in FIG. 5) may communicate data with each other, as well as a centralized server by means of ad-hoc mesh, or cellular wireless communication network. In particular the communications of individual communication nodes 501 with the centralized server may be facilitated by a stationary base-station 503, or a mobile base-station 502 that in some embodiments can take a form of an unmanned aerial vehicle, or a satellite.

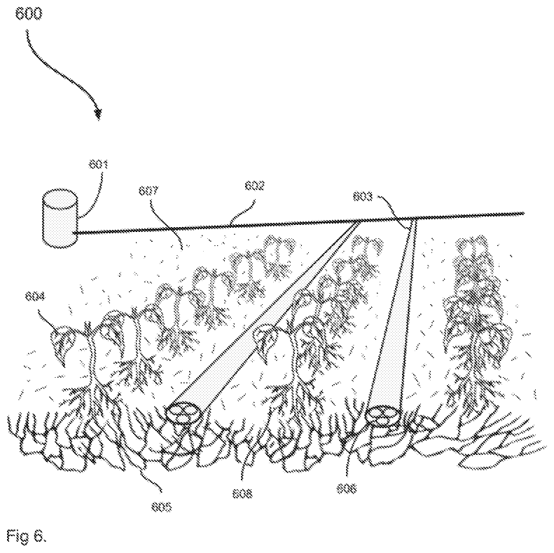

An agricultural input delivery system that performs targeted delivery of fertilizers and crop protection chemicals to a field of agricultural crops utilizing a network of bioelectrochemical reactors organized into an interconnected network is further illustrated in FIG. 6. Specifically, the disclosed system comprises a chemical container 601; main chemical distribution pipe 602; as well as a plurality of infield chemical distribution sub-pipes 603. Furthermore, each of the aforementioned sub-pipes 603 contains a plurality of containers 606. Each container 606 is filled with a substrate that is suitable to support the establishment of a mycelium network 605 also known as fungal tissue 104 (reference 104 not shown in FIG. 6). The resultant mycelium network 605 facilitates the exchange of nutrients and biochemical signals between the containers 606 and the root systems 608 of the plurality of plants 604. The resultant system 600 allows for the targeted delivery of fertilizers and crop protection chemicals from the central container 601 and the field plants 604. Furthermore, the said resultant system 600 allows for the registration of phenotyping sensory information recorded by the plurality of sensors embedded in the pipes 603 (not shown in FIG. 6) and detailed in FIG. 2 with the aim of optimization of the delivery of fertilizers and crop protection chemicals by system 600.

The various applications of the present plant system 100 comprise, but are not limited to uses in agriculture, forestry, environmental engineering and ecological monitoring.

It has been found that the plant releases specific chemical or biochemical signals corresponding to biotic or abiotic stresses and/or other physiological condition and phenotypic state such as maturity of the crops, flowering time etc.

In preferred embodiments
biotic stress may include the infestation with insects, fungi, nematodes, viruses, or bacteria;
abiotic stress may include drought, heat, cold, water, lack of nitrogen, lack of micronutrients; and
other phenotypical conditions may include flowering time, seed setting, seed water content, seed maturity etc.

The following table 1 lists examples of physiological condition/phenotypic state of plants, and the ensuing chemical/biochemical signal released. It also lists for each case, example sensor types to detect the condition/state and possible action(s) (or agronomic operations) to be carried out in response to the diagnosed condition/state. The actions may be notified to a user, who then takes appropriate action to address the condition/state. In a preferred embodiment the actions may trigger an automated process aimed at addressing the condition/state.

TABLE 1
Exemplary physiological conditions processed by the system of the invention

| Physiological | | | |
|---|---|---|---|
| condition/ | Chemical or | | |
| phenotypic stte | biochemical signal | Sensor | Action |
| Biotic stress | Strigolactones and | SL biosensors, | Release of biocontrol |
| including pressure | plant hormones | fluorometers, | agents including herbicides, |
| by competing, | involved in | MEMS | pesticides, nematicides, |
| parasitic or harmful | interplant, as well | sensors | fungicides, or other |
| plants, pests, fungi | as symbiotic and | | bioactive compounds |
| or microorganisms | parasitic | | |
| | interactions | | |
| Abiotic stress | nitrate reductase, | pH sensor; | Irrigation; Application of |
| including heat, cold, | glutamate | amperemeter; | strigolactones and Myc |
| drought, flooding, | synthetase, | MEMS | factors to promote root |
| salinity, acidification, | glutamate synthase, | sensors | development and AM |
| soil compaction and | ethylene, jasmonic | | symbiosys |
| physical damage | acid, salicylic acid, | | |
| | and strigolactones | | |
| Nutrient deficiency | pH gradient, | pH sensor; | Targeted distribution of |
| including | electrical current, | amperemeter; | macro and micro nutrients, |
| phosphorus (P), | strigolactones, | MEMS | including phosphorus (P), |
| nitrogen (N), | phosphatases, | sensors | nitrogen (N), potassium (K), |
| potassium (K), | genistein, | | calcium (Ca), magnesium |
| calcium (Ca), | lipochitooligosaccha | | (Mg), iron (Fe), manganese |
| magnesium (Mg), | rides | | (Mn), zinc (Zn), and copper |
| iron (Fe), | | | (Cu) |
| manganese (Mn), | | | |
| zinc (Zn), and | | | |
| copper(Cu) | | | |
| Plant phenology | Plant hormones | SL biosensors, | Agronomic operations |
| including maturity, | | fluorometers, | including topping, |
| leaf senescence, | | MEMS | application of growth |
| flowering, seed | | sensors | regulators, harvesting |
| protein content, | | | |
| seed water content | | | |

In an example embodiment, the sensor is preferably a miniature, portable mass-spectroscope as described—for example—in Sanders et al. (2010):

Additionally, examples of commercially available portable or hand-held devices to be used as a basis for sensors are commercially available—for example—from the following providers: DetectaChem (https://www.detectachem.com/); PKI Electronic Intelligence (http://www.pki-electronic.com/), smiths detection (https://www.smithdetection.com/prodcat-explosives-narcotics-tract-detection/) Westminster International Ltd (https://www.wi-ltd.com/solution/explosives-and-narcotics-detectors-and-analysers/), FILR systems (https://www.flir.com/threat-detection/explosives/) and others.

Commercially available devices as the ones available at above providers, are usually calibrated to detect chemicals like explosive or drugs, but can be easily calibrated by the person skilled in the art to detect any of the above chemical signals.

In preferred embodiments these signals are intercepted using a plurality of embedded sensors, allowing for the corresponding agronomic operations/action(s) to be carried out.

The system and method of the invention can be employed to any plurality of plants and are not limited to specific species. They can be applied to plants selected from the group consisting of annual, and perennial plants. Preferred perennial plants are trees (including fruit and forestry trees), coffee, cacao, banana, wine, berries, citrus, cassava, sugar cane and the like.

In a preferred embodiment the plant is an annual plant, more preferred an annual field crops, which can be selected from the group consisting of maize, soy, wheat, rice, barley, rapeseed, canola, tobacco and sunflower. Other preferred annual plants are vegetables, which can be selected from the group of tomato, pepper, melon, squash, cucumber, pumpkin, peas, beans, broccoli, cauliflower, cabbage, and Brussel sprouts.

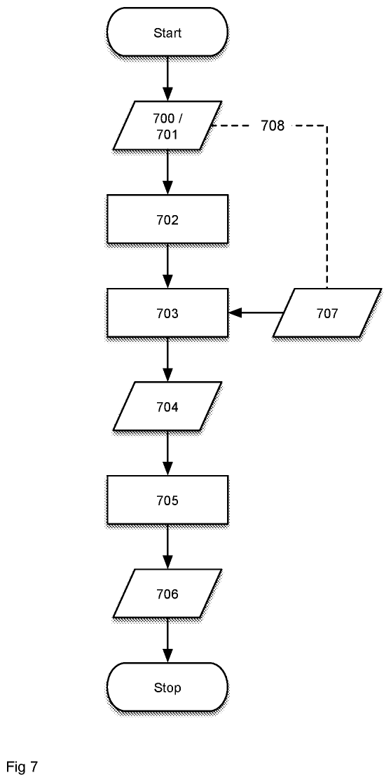

Another embodiment of the invention relates to a method for improving the physiological state of a plurality of plants. Referring to the flowchart in FIG. 7, said method comprises the steps of
(iv) planting 702 a plurality of plants 700 in a substrate 701 which comprises a mycorrhizal fungal community arranged to form a biological interface with roots of the plants enabling an exchange of chemical substances between the fungus and the plurality of plants; and
(v) collecting 703 sensory information 704 about a physiological condition and phenotypic state of said plurality of plants 700 using at least one sensor 707, which interfaces 708 with said mycorrhizal fungal community, and
(vi) analyzing 705 said collected sensory information 704 to trigger or suggest an action 706 to improve the physiological state of said plurality of plants.

In a preferred embodiment the sensory information are analyzed to detect biotic stress, abiotic stress, nutrient deficiency or plant phenology. Preferably the biotic stress is selected from the group comprising pressure by competing, parasitic or harmful plants, pests, fungi or microorganisms. Preferably the abiotic stress is selected from the group comprising heat, cold, drought, flooding, salinity, acidification, soil compaction and physical damage. Preferably, the nutrient deficiency is selected from the group comprising phosphorus (P), nitrogen (N), potassium (K), calcium (Ca), magnesium (Mg), iron (Fe), manganese (Mn), zinc (Zn), and copper (Cu) deficiency. Preferably the plant phenology is selected from the group comprising maturity, leaf senescence, flowering, seed protein content, and seed water content.

The sensory information is established by measuring chemical, biochemical compounds, or other signals transported or conveyed by the mycorrhizal fungal community.

Preferred biochemical signals are the detection of nitrate reductase, glutamate synthetase, glutamate synthase, Myc factors, or phosphatases. Preferred chemical signals comprise the detection of strigolactones, plant hormones, ethylene, jasmonic acid, salicylic acid, genistein, lipochitooligosaccharides. Preferred other signals comprise physical or electrochemical signals like pH gradient, or electrical current.

The person skilled in art is aware of suitable sensors to detect and measure these signals including but not limited to SL biosensors, fluorometers, MEMS sensors, pH sensor, and amperemeter.

Measurement and analysis can be conducted continuously or discontinuously. If the process is preformed discontinuously, samples can be taken from the mycorrhizal fungus community comprising substrate and analyzed for the relevant sensory information. If the process is performed continuously, the sensor is placed in the substrate to detect the relevant sensory information transported or conveyed by the mycorrhizal fungus community.

The signal is in general compared with a standard to detect a deviation from a preferred physiological state of the plant. This comparison can be established by a computer assisted system. This analysis preferably results is a recommendation for a specific action or—preferably—the system triggers the action automatically. The triggered or recommended action can comprise multiple actions known by the person skilled in the art to compensate for a deficiency in a plant. The action may—for example—include
(i) release of biocontrol agents selected from the group consisting of herbicides, pesticides, nematicides, fungicides, or other bioactive compounds;
(ii) Irrigation;
(iii) Application of strigolactones AM symbiosys;
(iv) Targeted distribution of macro and micro nutrients selected from the group consisting of phosphorus (P), nitrogen (N), potassium (K), calcium (Ca), magnesium (Mg), iron (Fe), manganese (Mn), zinc (Zn), and copper (Cu);
(v) Agronomic operations selected from the group consisting of topping, application of growth regulators, harvesting

The invention further provides a software product, which, when loaded and executed by a computer, enables the computer to implement the method steps of any one of the methods described herein above.

### FURTHER PREFERRED EMBODIMENTS

In a further preferred embodiment, the invention provides a bioelectrochemical plant-computer interface system, comprising a bioelectrochemical reactor configured to enable a controlled exchange of chemical substances with a plurality of plants; at least a sensor configured to collect sensory information about a physiological condition and phenotypic state of the plurality of plants; and at least a bioelectrochemical module configured to generate electrical current for powering the bioelectrochemical plant-computer interface system.

In a further preferred embodiment, the system further comprises a substrate enabled to support growth and development of at least one of the plurality of plants. The substrate is one of the list comprising at least a substrate enclosed in an artificial container; a substrate that forms a part of an agricultural field; and a natural substrate that is part of a natural vegetative habitat.

In a further preferred embodiment, the substrate is inoculated with mycorrhizal fungal community configured to permeate parts, or the entirety of a system's usable volume.

In a further preferred embodiment, the mycorrhizal fungal community is arranged to form a biological interface with roots of the plants enabling an exchange of chemical substances between the fungus and the plurality of plants.

In a further preferred embodiment, the bioelectrochemical reactor comprises at least one semi-isolated internal container that contains at least a chemical substance that is of physiological or nutritional significance to the plurality of plants.

In a further preferred embodiment, the bioelectrochemical reactor comprises at least one semi-isolated internal container that contains at least a chemical substance that is of physiological or nutritional significance to the plurality of plants; and the mycorrhizal fungal community forms a mycelium fiber network that permeates the container and carries the at least one chemical substance to the roots of a plurality of plants.

In a further preferred embodiment, the system further comprises a computational and communication device configured to record, process and communicate a plurality of analog and digital electrical signals; the computational and communication device comprises a radio interface enabled to transmit digital information to intended other computational devices, or an intended centralized base-station.

In a further preferred embodiment, at least one sensor is connected to the computation and communication device by means of conductive wires. The results of collected sensory information are transmitted to the computational and communication device in the form of analog or digital electric signals.

In a further preferred embodiment, a plurality of the at least one internal containers form a biochemical power cell that generates electric current that is utilized to power computational and communication devices and at least one sensor. A first of the plurality of internal containers is configured to generate an excess of cations and free electrons through biochemical reaction involving hyphae of the mycorrhizal network. A second of the plurality of internal containers is configured to absorb cations and free electrons through biochemical reaction involving hyphae of the mycorrhizal network. The system further comprises a cation transport membrane that separates the first internal container and the second internal container and is enabled to allow for the transport of ions between the two respective first and second internal containers; and a pair of electrodes connected by a conductive wire and a load that allows for a transport of electrons between the first and second internal containers.

A bioelectrochemical system as described in the present section comprises a substrate that is able to support the growth and development of one or multiple plants. In one embodiment, an example of the substrate is a natural fertile soil. The substrate is either enclosed in an artificial container, or is open and constitutes a section of an agricultural field, or a natural vegetative ecosystem.

The substrate is inoculated with mycorrhizal fungus community that permeates part or the entirety of its volume. In particular, a mycelium network produced by the mycorrhizal fungus community creates a biological interface with roots of the plants enabling an exchange of chemical substances between the mycorrhizal fungus community and the plant.

The bioelectrochemical system further comprises a bioelectrochemical reactor that enables the controlled exchange of chemical substances with an hyphae of the mycelium network.

The bioelectrochemical system further comprises a computing device that is capable to record, process and communicate a plurality of analog and digital electrical signals transmitted by the bioelectrochemical reactor. In particular, the computational device comprises a radio interface that is able to transmit digital information to other computational devices, or a centralized base-station.

The bioelectrochemical system further comprises a power module that may be configured to collect and store electrical energy generated by the bioelectrochemical reactor. Subsequently, the power module may be configured to supply electrical energy to empower the operation of the computing device.

No language in the specification should be construed as indicating any non-claimed element as essential to the practice of the invention.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiments disclosed, but that the present disclosure will include all embodiments falling within the scope of the appended claims.

### REFERENCES (INCORPORATED BY REFERENCE)

AU2017201009, Indigo Agriculture, Inc. Endophytes, associated compositions, and methods of use thereof.
US20170107160, Newman D. K., Kulkarni G., Belin B. J. Mar. 7, 2017.
US 2005/0132431 A1
WO 2012/038740 A1
US20170020138, Von Maltzahn G., Flavell R. B., Toldeo G. V., Jack A., Johnston D. M., Djonovic S., Marquez L. M., Millet Y. A., Sadowski C., Lyford J., Naydich A. Hopanoids producing bacteria and related biofertilizers, compositions, methods and systems.
PCT/US2016/043933, Wigley P., Turner S., George C., Wright D., Williams T., Roberts. K, Hymus G. Agriculturally beneficial microbes, microbial compositions, and consortia.
Chen W., Koide R. T., Adams T. S., DeForest J. L., Cheng L., Eissenstat D. M., 2016. Root morphology and mycorrhizal symbioses together shape nutrient foraging strategies of temperate trees. PNAS 113: 8741-8746.
Zahran H. H. 1999. Rhizobium-legume symbiosis and nitrogen fixation under severe conditions and in an arid climate. Microbiology and Molecular Biology Review. 63:968-89.
Aroca R. ed., 2013. Symbiotic Endophytes, Springer-Verlag.
Samodelov S. L. et al., 2016. StrigoQuant: A genetically encoded biosensor for quantifying strigolactone activity and specificity, Science Advances. DOI: 10.1126/sciadv.1601266.
Jackson, T. et al., 2008. Measuring soil temperature and moisture using wireless MEMS sensors, Measurement, 41(4).
Rout, B. et al., 2013. Combinatorial Fluorescent Molecular Sensors: The Road to Differential Sensing at the Molecular Level, SYNLETT, 25.
Sanders, N L. et al., Detection of Explosives as Negative Ions Directly from Surfaces Using a Miniature Mass Spectrometer; Department of Chemistry, Purdue University, West Lafayette, Ind. 47907-2084; Anal. Chem., 2010, 82 (12), pp 5313-5316; DOI: 10.1021/ac1008157; https://pubs.acs.org/doi/10.1021/ac1008157.

1. A plant system, comprising
(i) a plurality of plants in a substrate;
(ii) a mycorrhizal fungal community in the substrate arranged to form a biological interface with roots of the plants enabling an exchange of chemical substances between the fungus and the plurality of plants; and
(iii) at least a sensor, which interfaces with said mycorrhizal fungal community and is configured to collect sensory information about a physiological condition and phenotypic state of said plurality of plants.

2. The plant system of claim 1, wherein the sensor is continuously interfacing with the mycorrhizal fungal community and able to detect chemical or biochemical substances without disrupting said community.

3. The plant system of claim 1, wherein the sensor is based on a method selected from a list consisting of mass spectroscopy, Raman spectroscopy, SL biosensor, fluorometer, MEMS sensor, pH sensor, amperemeter.

4. The system of any of claim 1, wherein the sensor is linked to a computer converting the sensory information into a recommendation for action or action.

5. The plant system of claim 4, wherein the action is selected from the group consisting of treating the plants with chemical or biochemical substances, irrigation, harvesting, release of biocontrol agents such as any one from the list comprising herbicides, pesticides, nematicides, fungicides, application of strigolactones and Myc factors, targeted distribution of macro nutrient, targeted distribution of micro nutrient such as any one from the list comprising phosphorus (P), nitrogen (N), potassium (K), calcium (ca), magnesium (Mg), iron (Fe), manganese (Mn), zinc (Zn), copper (Cu), topping, application of growth regulators.

6. The system of claim 1, further comprising a reactor configured to enable a release of chemical or biochemical substances into the mycorrhizal fungal community to the plurality of plants rooted in a substrate.

7. The system of claim 1, further comprising at least a bioelectrochemical module configured to generate electrical current for powering a bioelectrochemical plant-computer interface system.

8. The system of claim 1, wherein the substrate is enabled to support growth and development of at least one of the plurality of plants; and
whereby the substrate is one of the list comprising at least
a substrate enclosed in an artificial container;
a substrate that forms a part of an agricultural field; and
a natural substrate that is part of a natural vegetative habitat.

9. The system of claim 1, wherein the substrate is inoculated with mycorrhizal fungal community configured to permeate parts, or the entirety of a system's usable volume.

10. The system of claim 6, wherein the reactor is a bioelectrochemical reactor comprising at least one semi-isolated internal container that contains at least a chemical substance that is of physiological or nutritional significance to the plurality of plants.

11. The system of claim 11, wherein the bioelectrochemical reactor comprises at least one semi-isolated internal container that contains at least a chemical substance that is of physiological or nutritional significance to the plurality of plants; and the mycorrhizal fungal community forms a mycelium fiber network that permeates the container and carries the at least one chemical substance to the roots of a plurality of plants.

12. The system of claim 1, further comprising a computational and communication device configured to record, process and communicate a plurality of analog and digital electrical signals;
the computational and communication device comprising a radio interface enabled to transmit digital information to intended other computational devices, or an intended centralized base-station.

13. The system of claim 12, wherein
at least one sensor is connected to the computation and communication device by means of conductive wires;
the results of collected sensory information being transmitted to the computational and communication device in the form of analog or digital electric signals.

14. The system of claim 10, wherein a plurality of the at least one internal containers form a biochemical power cell that generates electric current that is utilized to power computational and communication devices and at least one sensor, and wherein
a first of the plurality of internal containers is configured to generate an excess of cations and free electrons through biochemical reaction involving hyphae of the mycorrhizal network;
a second of the plurality of internal containers is configured to absorb cations and free electrons through biochemical reaction involving hyphae of the mycorrhizal network;
the system further comprising
a cation transport membrane that separates the first internal container and the second internal container and is enabled to allow for the transport of ions between the two respective first and second internal containers; and
a pair of electrodes connected by a conductive wire and a load that allows for a transport of electrons between the first and second internal containers.

15. The system of claim 1, wherein the plurality of plants is comprising plants selected from the group consisting of annual, and perennial plants.

16. The system of claim 15, wherein the plant is an annual field crops selected from the group consisting of maize, soy, wheat, rice, barley, rapeseed, canola, tobacco and sunflower.

17. The system of claim 15, wherein the plant is a vegetable selected from the group consisting of tomato, pepper, melon, squash, cucumber, pumpkin, peas, beans, broccoli, cauliflower, cabbage, Brussel sprouts.

18. A method for improving the physiological state of a plurality of plants, said method comprising steps of
(i) planting a plurality of plants in a substrate which comprises a mycorrhizal fungal community arranged to form a biological interface with roots of the plants enabling an exchange of chemical substances between the fungus and the plurality of plants;
(ii) collecting sensory information about a physiological condition and phenotypic state of said plurality of plants using at least one sensor, which interfaces with said mycorrhizal fungal community, and
(iii) analyzing said collected sensory information to trigger or suggest an action to improve the physiological state of said plurality of plants.

19. The method of claim 18, wherein the sensory information is analyzed to detect biotic stress, abiotic, nutrient deficiency or plant phenology.

20. The method of claim 19, wherein
(i) the biotic stress is selected from the group comprising pressure by competing, parasitic or harmful plants, pests, fungi or microorganisms;
(ii) the abiotic stress is selected from the group comprising heat, cold, drought, flooding, salinity, acidification, soil compaction and physical damage;
(iii) the nutrient deficiency is selected from the group comprising phosphorus (P), nitrogen (N), potassium (K), calcium (Ca), magnesium (Mg), iron (Fe), manganese (Mn), zinc (Zn), and copper (Cu) deficiency; and/or
(iv) the plant phenology is selected from the group comprising maturity, leaf senescence, flowering, seed protein content, and seed water content.

21. The method of claim 18, wherein the sensory information is established by measuring chemical, biochemical compounds, or other signal transported or conveyed by the mycorrhizal fungal community.

22. The method of claim 21, wherein the biochemical, chemical compound, or other signal is selected from the group consisting of strigolactones, plant hormones, nitrate reductase, glutamate synthetase, glutamate synthase, ethylene, jasmonic acid, salicylic acid, Myc factors, genistein, lipochitooligosaccharides, phosphatases, pH gradient, or electrical current.

23. The method of claim 18, wherein the sensor is selected from the group consisting of SL biosensors, fluorometers, MEMS sensors, pH sensor; amperemeter

24. The method of claim 18, wherein the action is triggered automatically by a computer system.

25. The method of claim 18, wherein the triggered or recommended action is selected from the group consisting of
(i) release of biocontrol agents selected from the group consisting of herbicides, pesticides, nematicides, fungicides, or other bioactive compounds;
(ii) Irrigation;
(iii) Application of strigolactones AM symbiosys;
(iv) Targeted distribution of macro and micro nutrients selected from the group consisting of phosphorus (P), nitrogen (N), potassium (K), calcium (Ca), magnesium (Mg), iron (Fe), manganese (Mn), zinc (Zn), and copper (Cu);
(v) Agronomic operations selected from the group consisting of topping, application of growth regulators, harvesting.

26. A software product which when loaded and executed by a computer, enables the computer to implement the method steps of claim 18.
